(19) United States
(12) Patent Application Publication
(10) Pub. No.: US 2010/0198095 A1
(43) Pub. Date: Aug. 5, 2010

(54) DEVICE,GAS MIXTURE AND METHOD FOR LUNG DIAGNOSIS

(75) Inventor: Rudolf Isler, Hinteregg (CH)

Correspondence Address: THE NATH LAW GROUP, 112 South West Street, Alexandria, VA, 22314, US

(73) Assignee: ECO MEDICS AG, Durnten (CH)

(21) Appl. No.: 12/733,529

(22) PCT Filed: Sep. 5, 2008
(86) PCT No.: PCT/CH2008/000371

§ 371 Date: Mar. 5, 2010

(30) Foreign Application Priority Data
Sep. 7, 2007 (CH) 140707

(51) Int. Cl. A61B 5/08; C09K 3/00

(52) U.S. Cl. 600/532; 252/372

(57) ABSTRACT

The invention relates to a lung diagnostic device, namely one for determining the ventilation homogeneity (VH) of a spontaneously breathing or artificially respirated patient as well as a gas mix for use in such a device. In this respect the difference in the composition of the exhaled test gas from the composition of the inhaled test gas serves for the evaluation of the ventilation homogeneity.

The invention relates to a lung diagnostic device, a test gas mix suitable therefore as well as a method for determining the ventilation homogeneity (VH) of a spontaneously breathing or artificially respirated patient.

For measuring parameters of the lung function, amongst others, methods are used, which can measure the in- and exhaled volumes as precisely as possible and without discomfort to the patients. With known systems the breathing volumes are most often determined by sensors, which determine the median flow rate (flow) in a known area cross section of the sensor.

In particular, known devices used for this purpose encompass differential pressure analyzers, e.g. pneumotachographic devices, turbines, thermistors, vortex flow meters and the ultrasound runtime method.

The referenced devices have different disadvantages which limit their applications, in particular a too limited linear measurement range, a dependency on the gas composition and temperature, a too high instrument shunt, an adverse frequency behaviour, a problematic sterilizing characteristic, as well as a too elaborate and/or too often necessary calibration. Also, cooperation of the patient is not always guaranteed.

In the context of investigations on breathing disorders in cystic fibrosis (CF) the use of gas mixtures with a content of inert but relatively specifically heavier gases such as sulphur hexafluoride has been suggested for determining the ventilation distribution in the lung (Van Mullen, A. et Baren, D., Pediatric Pulmonology 30:30-9 (2000)), in particular in the expectation that different distributions of the differently heavy gas components will change more intensely in pathological lungs (COPD, CF). Furthermore, it is known that the molecular mass sum (MMS) of the inspiriation and exspiration gas mix can be measured directly in the respiratory flow by means of ultrasound measurement (Buess, Ch. et al, IEE Trans. Biomed. Eng., 33(8) 768-774, 1986)).

It is the object of the invention to determine the ventilation homogeneity (VH) of a spontaneously breathing or artificially respirated patient in a simple metrological manner. According to a first embodiment of the invention this object is solved by a device having the features of claim 1, i.e. a device for determining the ventilation homogeneity of a spontaneously breathing or artificially respirated patient with at least one source for a breathable test gas mix (also simply designated as test gas) having essentially the same molecular mass sum as ambient air or a reference gas mix, but which differs from both by addition of at least one specifically heavier inert gas and optionally at least one specifically lighter inert gas for compensating the molecular mass sum, and a unit for determining the molecular mass sums of the test gas mixture exhaled by the patient.

The reference gas mix or reference gas used instead of ambient air is a breathable and pharmacologically acceptable gas or gas mixture with a sufficient oxygen content between that of ambient air and 100 vol.-%, optionally with the addition of a specifically lighter gas such as helium for adapting the molecular mass sum of the reference gas to that of ambient air.

Preferred embodiments of the device according to the invention have the features mentioned in claims 2 to 10.

According to a second embodiment the invention provides a test gas mix having the features mentioned in claim 11, i.e. a test gas mix for determining the ventilation homogeneity of a spontaneously breathing or artificially respirated patient, namely a breathable gas mix, which has essentially the same molecular mass sum as the ambient air, but differs therefrom by at least one added specifically heavier inert gas and optionally (i.e., if or because the reference gas mix or ambient air already comprises a specifically lighter inert gas in a more or less large proportion) of a specifically lighter inert gas.

Preferred embodiments of the test gases have the features mentioned in claims 12 to 16.

According to a further embodiment the invention provides a method having the features mentioned in claim 17, i.e. a method for determining the ventilation homogeneity of a spontaneously breathing or artificially respirated patient, wherein the patient inhales actively or passively and in a controlled manner a breathable test gas mix, which essentially has the same molecular weight sum as ambient air or a reference gas mix, but differs from both by addition of at least one specifically heavier inert gas and optionally at least one specifically lighter inert gas, and wherein the molecular mass sum (MMS) of the test gas exhaled by the patient is determined in order to evaluate the ventilation homogeneity of the patient from the differences in the molecular mass sums (MMS) of the test gas mix inhaled and exhaled by the patient.

Despite its content of relatively and specifically heavier components the test gas mix according to the invention overall has essentially the same molecular mass sum as normal breathing air or a reference gas mix, the same breathability as normal ambient air, because the content of the specifically heavier gas is compensated by the content of the specifically lighter gas.

Relative to the specifically heavier components the specifically lighter components of the test gas mix disperse faster in the lung. In this way the different time constants for pathological lung indications (e.g. COPD, CF) can be determined better, more reliably and simpler, because the relatively short-term changes in the composition of the exhaled air can be determined by known methods such as ultrasound measurement, when the test gas mix comprising the specifically heavier component has essentially the same molecular mass sum as the ambient air or the reference gas mix without a specifically heavier component.

In general, a breathable gas mix is one, wherein oxygen is comprised in a sufficient content of preferably at least 21% or more and which is pharmacologically acceptable and otherwise inert to the lungs.

If instead of ambient air a breathable gas mix without the specifically heavier component is used as reference gas mix, this mix can comprise oxygen in a concentration of 21 to 100, preferably 30 to 100, more preferably 50 to 100, most preferably 100 vol.-%, and optionally a specifically lighter component such a helium for compensating the increased molecular mass sum due to the increased oxygen content. In general, a reference gas mix is used instead of ambient air, when an increased oxygen content is medically advisable or necessary for artificial respiration.

Preferably the molecular mass sum of the test gas mix and the reference gas mix or the reference gas differs by less than 10%, preferably by less than 5%, more preferably by less than 2%, most preferably by less than 1% from the molecular mass sum of the ambient or the reference gas mix.

The test gas mix as well as the reference gas or the reference gas mix optionally used instead of ambient air preferably has a molecular mass sum of 28 to 33 g/mol, preferably about 29 or 32, more preferably 28.85 or 32.0 g/mol (e.g. in the case of normal air of 100% oxygen).

In a very preferred embodiment the at least one specifically heavier inert gas (I2) in the test gas mix is selected from the group consisting of argon, neon, krypton, radon, xenon and SF₆ as well as mixtures thereof, preferably radon and SF₆ and for technical measurement reasons more preferably SF₆.

The at least one specifically lighter inert gas in the test gas mix or optionally in the reference mix is helium.

For determining the molecular mass sum (MMS) of the test gas mix exhaled by the patient (P) preferably a device for measuring the ultrasound speed in gases or gas mixtures and a unit for calculating the molecular mass sum from the measured ultrasound speeds as well as a unit for displaying the measured values.

The device of the invention can comprise a second source for the reference gas mix, which preferably has a higher oxygen content than ambient air, no addition of the specifically heavier gas and optionally the addition of a specifically lighter inert gas such as helium for compensating an increased oxygen content.

If desired, the unit for determining the molecular mass sum is a unit for calculating the measured values for standard conditions (normalising).

For functional reasons, the device according to the invention is designed so that it can switch between the source for the test gas mix and a source for the reference gas mix or ambient air.

The diagnosis method performed according to the invention is very generally based on the measurement of the molecular mass sum (MMS) of gas mixtures and a difference between the molecular mass sum of the inhaled and exhaled gas mix. The reason is the different diffusion rate of both gas components, which based on the different collision cross sections and because of the different molar masses changes the median free path length. The MMS for ambient air is about 28.85 g/mol (+0.1 g/mol depending on temperature and humidity). The skilled person will understand that other mathematical derivatives derived directly from the MMS, e.g. MMS relative to the breathing volume, CO₂ content, etc. are preferably also encompassed by the term MMS.

The necessary measurements can be done without any problems by means known for other applications. Suitable measurement units for practicing the invention are, e.g. devices for measuring the velocity of propagation of sound and ultrasound waves in flowing medium, devices for determining the thermal conductivity or the light or UV or IR adsorption as well as electroacoustic analyzers.

A more preferred device according to the invention for determining the molecular mass sum of the used gas mix is based on the measurement of absolute sound delay times in the main and/or side stream, in particular ultrasound (US) delay times, for measuring the instant breath flow rate, and thereby the breath flow volumes. This allows for a high linearity, an independency from the gas composition and temperature, a high spatiotemporal resolution as well as very good hygiene characteristics.

Units for the ultrasound runtime analysis further offer the possibility of a simultaneous determination of the mole mass of flowing gases or the median molar mass of flowing gas mixtures as well as information about the gas flow and gas composition in real-time.

The terms “specifically heavier” and “specifically lighter” as used herein for the characterization of gas components relate to the specific weight of the main components of breathing air, nitrogen and oxygen. Because in a breathable gas mixture nitrogen can be replaced by other inert gases, whereas oxygen is critical for breathability, the above mentioned terms are based on oxygen.

Test gas mixes according to the invention and reference gas mixes are homogenous mixtures with a defined composition, the accuracy of which should generally be better than 0.1% (+/−0.02 g/mol). Naturally, the purity required for medical purposes (e.g. p.a.) must be guaranteed.

The term “molecular mass sum” (MMS) relates to the molecular mass resulting from the relative proportions of the components and their respective molecular masses. For example, the molecular mass sum of a mixture of 50% oxygen (molecular mass of 32) and 50% helium (molecular mass 4) yields 36/2=18.

The terms “reference gas mix” or “reference gas” relate to a breathable gas having the same molecular mass sum as the test gas mix but containing no specifically heavier component and comprising the specifically lighter component in the case, when the reference gas mix, as most often preferred, should have the same molecular mass sum as the ambient air.

The terms “essentially”, “nearly” or “about” antecedent a numerical value mean herein a deviation of ±10%, preferably ±5%, more preferably ±2%, preferably less than ±1%. If not mentioned otherwise, Data in percent with regard to gas mixtures relates to the volume.

The operation of the device or the method of the invention requires no cooperation of the patient as for other conventional measurements of lung function. Hence, the device and method are particularly well-suited for coma patients and newborns. Additionally to the above-mentioned general and specific indications exemplary and preferred applications of methods and devices according to the invention are in particular also in the following fields:

Objectifying ventilation disorders (obstructive, restrictive, combined);

Testing the reversibility of distribution disorders (bronchospasmolysis test);

Provocation of ventilation disorders (unspecific or specific provocation test);

Progression parameters of COPD (chronic obstructive pulmonary disease);

Early diagnosis of CF (cystic fibrosis; as progression parameter);

Evaluation of effects of medicaments on distribution disorders.

In the following air (table 1, gas A) is compared to a test gas mix according to the invention with essentially the same MMS (table 2, gas B) as a preferred embodiment in a tabular manner.

TABLE 1

| air | | | |
|---|---|---|---|
| gas A | atomis mass | % gas | MMS |
| O | 15.9994 | | 0.00 |
| O₂ | 31.9988 | 21.00 | 6.72 |
| He | 4 | 0.00 | 0.00 |
| N | 14.0067 | | 0.00 |
| N₂ | 28.0134 | 79.00 | 22.13 |
| S | 32.064 | | 0.00 |
| F | 18.9984 | | 0.00 |
| SF₆ | 146.0544 | 0.00 | 0.00 |
| Total | | 100.00 | 28.85 |

TABLE 2

| test gas | | | |
|---|---|---|---|
| gas B | atomic mass | % gas | MMS |
| O | 15.9994 | | 0.00 |
| O₂ | 31.9988 | 21 | 6.72 |
| He | 4 | 29.5 | 1.18 |
| N | 14.0067 | | 0.00 |
| N₂ | 28.0134 | 43.5 | 12.19 |
| S | 32.064 | | 0.00 |
| F | 18.9984 | | 0.00 |
| SF₆ | 146.0544 | 6 | 8.76 |
| Total | | 100 | 28.85 |

In the following a reference gas (gas C) of essentially pure oxygen is shown together with a test gas according to the invention (gas D) of the same MMS as a further example of a test gas/reference gas pair.

TABLE 3

| reference gas | | | |
|---|---|---|---|
| gas C | atomic mass | % gas | MMS |
| O | 15.9994 | | 0.00 |
| O₂ | 31.9988 | 100.00 | 32.00 |
| He | 4 | 0.00 | 0.00 |
| S | 32.064 | | 0.00 |
| F | 18.9984 | | 0.00 |
| SF₆ | 146.0544 | 0.00 | 0.00 |
| Total | | 100.00 | 32.00 |

TABLE 4

| test gas | | | |
|---|---|---|---|
| gas D | atomic mass | % gas | MMS |
| O | 15.9994 | | 0.00 |
| O₂ | 31.9988 | 65.00 | 20.80 |
| He | 4 | 28.10 | 1.12 |
| S | 32.064 | | 0.00 |
| F | 18.9984 | | 0.00 |
| SF₆ | 146.0544 | 6.90 | 10.08 |
| Total | | 100.00 | 32.00 |

In the following table 5 a preferred test gas mix according to the invention of helium, radon and oxygen having essentially the same MMS as ambient air is shown.

TABLE 5

| components | atomic mass | % content | MMS |
|---|---|---|---|
| O₂ | 31.9988 | 21 | 6.72 |
| He | 4 | 26.7 | 1.07 |
| CO₂ | 35.9988 | 0 | 0.00 |
| N₂ | 28.0134 | 49 | 13.73 |
| S | 32.064 | | 0.00 |
| F | 18.9984 | | 0.00 |
| SF₆ | 146.0544 | 0 | 0.00 |
| neon | 20.17 | 0 | 0.00 |
| argon | 39.94 | | 0.00 |
| krypton | 83.8 | | 0.00 |
| xenon | 131.2 | 0 | 0.00 |
| radon | 222 | 3.3 | 7.33 |
| Total | | 100 | 28.84 |

The skilled person will recognize that the knowledge of the resting breathing state is essential for the interpretation of the measurement results. The breathing gases CO₂ and O₂, and/or also the breathing volumes by the minute provide essential information about the resting breathing state and can be used as reference points for the interpretation of the measurement data.

In order to assist the patients to breathe in their optimum resting breathing state, an “incentive screen” or an “animation device” with or without direct “biofeedback” display represents a further interesting application of the present invention.

The invention will now be illustrated in four figures as well as by non-limiting examples. In the figures:

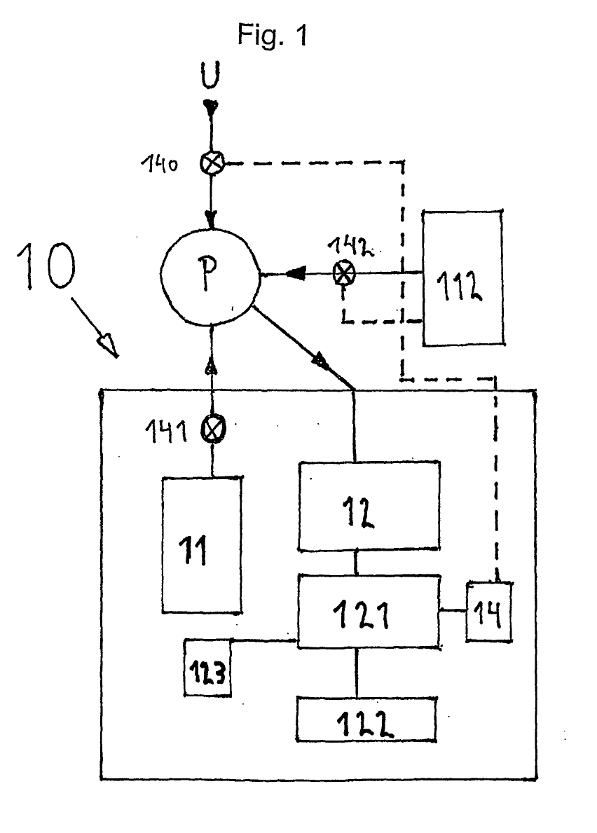

FIG. 1 shows the diagram of a device according to the invention;

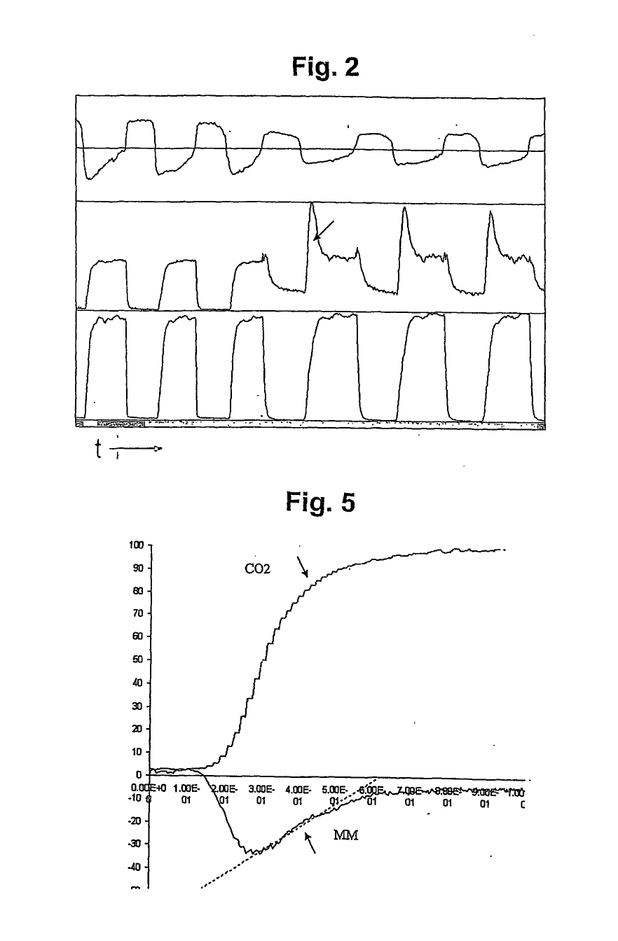

FIG. 2 shows three data curves of twice three breaths presented together with different measurement parameters on the X-axis and time on the y-axis;

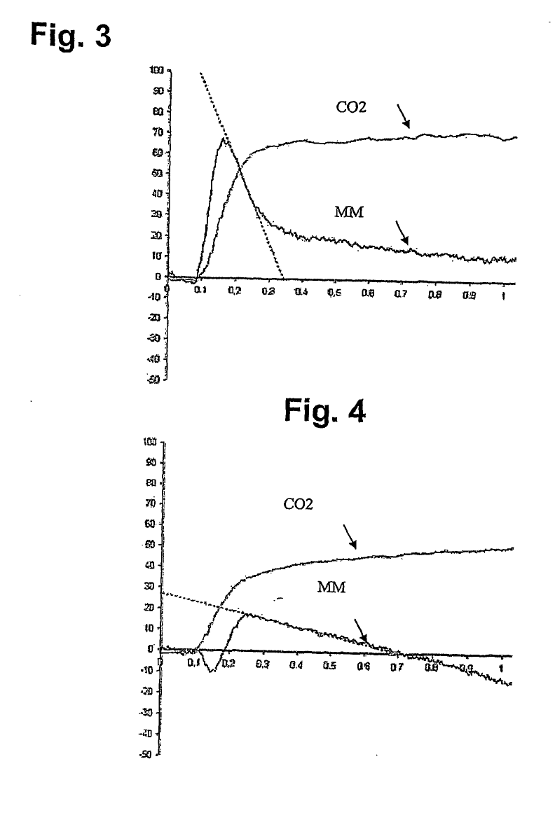

FIG. 3 shows the data curves of a patient with normal ventilation homogeneity, wherein the molecular mass sum in percent of the minimum and maximum values is plotted on the x-coordinate and the breathing volume in liters on the y-coordinate;

FIG. 4 shows the analogous data curves as in FIG. 3 but for a COPD patient with disordered ventilation homogeneity;

FIG. 5 shows the change in the molecular mass sum of the first expiration of reference gas after a complete wash-in procedure of test gas.

The device 10 depicted schematically in FIG. 1 has at least one source 11 for a breathable gas mix of the invention which practically has the same molecular mass sum as the ambient air but differs therefrom by the addition of at least one specifically lighter inert gas such as helium and at least one specifically heavier gas such as sulfur hexafluoride and at least one specifically heavier inert gas; and at least one unit 12 for determining the molecular mass sum of the gas mix of the invention and the ambient air exhaled by the patient.

Additionally to source 11 the device 10 can feature for the gas mix of the invention a reference gas mix from a second source 12. This gas mix replaces the ambient air (AA) if a higher oxygen content is necessary for breathing. Because the molecular mass sum (MMS) of this reference gas mix with an increased oxygen content is preferably the same as the MMS of the ambient air and the test gas mix of the invention, and because the increased oxygen content is appropriately compensated by a reduced nitrogen content, the influence of the increased content of the specifically heavier oxygen (than nitrogen) can be compensated by addition of a specifically lighter inert gas, in particular helium. This is redundant if the test gas mix has the same molecular mass sum as the reference gas.

The unit 12 for determining the molecular weight mass of the gas mix of the invention exhaled by the patient (P) is preferably a device for measuring the ultrasound speed in gases and gas mixtures. The unit 121 for calculating the measured ultrasound speeds into the molecular mass sum and the unit 122 for displaying the measured values can be part of the device of the invention 10 or be arranged separately. Moreover, the device 10 can comprise a well-known unit 123 for calculating the measured values relative to standard conditions.

According to a preferred embodiment the device 10 further comprises units 14; 140; 141; 142 for switching from the gas mix of ambient air (AA) inhaled by the patient (P) to the test gas mix of the invention delivered from source (11) and back; or for switching from the second gas mix from source 112 exhaled by the patient to the gas mix of the invention from source 11 and back.

In practice, for example, a commercially available device for other applications is used, such as the one available from the applicant under the trade marks Exhalyzer D® or Spiroson Scientific®, and which has two ultrasound (US) emitter and receiver units, which are positioned on the side of a breathing tube guiding the air flow. For determining the flow rate the following steps are followed: In a first measurement cycle a US pulse is emitted from the US emitter/receiver, runs through the measuring canal and thereby through the breathing stream and is consequently received by the opposite US-emitter/receiver. The runtime of the sound pulse is determined precisely by means of digital electronics (resolution 10 ns).

In a subsequent measuring cycle a US pulse is emitted in the opposite direction and its run time is determined. Because on its path the sound propagates one time with the gas flow and another time against the gas flow is differs in both run times.

For practical operation of the invention preferably a US emitter/receiver is employed as it is known from microphone technology and which enables the emission and the precise receiving of very short sound impulses. This allows for an optimum sound transfer and the measuring of the absolute sound run times. For calculations known means of digital technology can be used. By optimization of the emission/receiving electronic measurements of the US run times a resolution of 10 ns can be achieved.

Preferably the measurement is performed in guide tubes with small tube diameters typically in the range of 30 to 6 mm, wherein naturally the mechanical design of the measuring tube should correspond to the requirements of proper hygiene.

### Example 1

Gas 1 (atmospheric air) and gas 2 (gas mix of the invention) have an identical molecular mass sum of 28.9 g/mol.

The oxygen content of both gases is at least about 21%. The gases admixed to the gas mix of the invention (He/SF₆) are inert, i.e. they are not absorbed by the body. Instead of the normal atmospheric air a further gas mix with an increased oxygen content can be used when it is necessary for medical reasons to work with an increased oxygen content. The composition of gases 1 and 2 is specified in the following table 6.

TABLE 6

| | gas 1 | MM | gas 2 | MMS |
|---|---|---|---|---|
| | part % | (g/mol) | part % | (g/mol) |
| O₂ | 21 | 6.72 | 21 | 6.719748 |
| N₂ | 79 | 22.130586 | 32 | 8.964288 |
| He | | 0 | 39 | 1.561014 |
| SF₆ | | 0 | 8 | 11.684352 |
| Total | 100 | 28.9 | 100 | 28.9 |

During measurement the difference between the MMS (now dMMS) between the inspiration and the expiration is measured. If one switches from gas 1 (reference gas or ambient air) to gas 2 (test gas) the lighter and the heavier components of the gases are exhaled in a homogeneously ventilated lung in a relationship typical for a lung with homogenous ventilation. The MMS standard curve is well reproducible.

In a non-homogenously ventilated lung the ratio of the expiration gas mixture is more different, because the lighter helium (He) in the residual volume (the air volume in the lung, that is also left in the lung during forced exhalation) is dispersed faster than the heavier SF₆. The difference between this MMS-wash-out curve and the MMS standard curve can optionally be evaluated by calculation if desired.

For example, at the first breath of gas 2 about 50% He-content and 25% SF₆ content is left in the lung, i.e. 50% He-content and 75% SF6 content are measured in the expiration of the first breath and a strong MMS increase (see FIG. 3, arrow in the lower curve) compared to the CO₂ curve (arrow in the upper curve) is recognizable.

If the residual volume is also completely washed out with gas 2, one switches back to gas 1 and observes the reciprocal phenomenon—He is eliminated faster from the lung than SF₆ and one measures a strong decrease in MMS with the first breaths of gas 1 (see FIG. 5, arrow in the lower curve).

### Example 2

FIG. 2 shows an example of a measurement according to the invention of thee breaths gas 1 (air) and three breaths gas 2. (Flow (upper curve; inspiration ‘plus’, exspiration ‘minus’); MMS, SF₆/He (middle curve), CO₂ (lower curve)). For the exspiration the MMS signal for gas 1 is identical with the signal for CO₂, because CO₂ is heavier than air, i.e. the MMS signal corresponds essentially to the CO₂ signal. Because now the SF₆ (high MMS) disperses worse than helium (He) (low MMS) relatively more SF6 than He comes back from the first expiration with gas 2. Therefore, one recognizes a prominent increase in MMS (see arrow). This increase decreases continuously until the lung is washed to homogeneity.

### Example 3

FIGS. 3 and 4 are respective records of MMS versus expirated volume in a healthy human and a human with a lung disorder with the gas mix according to the invention. The upper arrow marks the CO₂ curve, the lower arrow marks the MMS curve of the first expirated breath with gas 2. When comparing a healthy lung (FIG. 3) with a COPD (chronic obstructive pulmonary disease) lung (FIG. 4) (chronic strong smoker), this increase in the expiration of the MMS curve of the first expirated breath with gas 2 is markedly different. This would explain that the different distribution of the differently heavy gases—which is important for the invention—is more prominent for badly ventilated lungs than for healthy lungs.

In general and for diagnostic purposes the results of a patient can be compared to the results of a patient with corresponding standard values of a healthy population or with the calculated theoretical or the previously measured values of the same patient.

For the skilled person many variations are evident within the context of the following patent claims in view of the examples, the description and the figures.

1. A device (10) for determining the ventilation homogeneity (VH) of a spontaneously breathing or artificially respirated patient (P), characterized in that it comprises:
at least one source (11) for a breathable test gas mix having essentially the same molecular mass sum (MMS) as ambient air (AA) or as a reference gas mix from a second source (112), but which differs from the ambient air (AA) or the reference gas mix from the second source (112) by addition of at least one specifically lighter inert gas (I1) and at least one specifically heavier inert gas (I2), and
at least one unit (12) for determining the molecular mass sums (MMS) of the gas mixtures exhaled by the patient.

2. The device (10) according to claim 1, characterized in that the molecular mass sum of the test gas mix differs by less than 10%, preferably by less than 5%, more preferably by less than 2%, most preferably by less than 1% from the molecular mass sum of the ambient air (AA) or the reference gas mix from a second source (112).

3. The device (10) according to claim 1, characterized in that the reference gas mix from the second source (112) comprises oxygen in a concentration of 21 to 100, preferably 30 to 100, more preferably 50 to 100, most preferably 100 vol.-%.

4. The device (10) according to claim 1, characterized in that the test gas mix has a molecular mass sum of 28 to 33 g/mol, preferably about 29 or 32, more preferably 28.85 or 32.0 g/mol.

5. The device (10) according to claim 1, characterized in that the at least one specifically heavier inert gas (I2) in the test gas mix is selected from the group consisting of argon, neon, krypton, radon, xenon and SF₆ as well as mixtures thereof, preferably radon and SF₆ and more preferably SF₆.

6. The device (10) according to claim 1, characterized in that the at least one specifically lighter inert gas in the test gas mix is helium.

7. The device (10) according to claim 1, characterized in that the unit (12) for determining the molecular mass sum (MS) of the test gas mix exhaled by the patient (P) has a device for measuring the ultrasound speed in gases or gas mixtures and a unit (121) for calculating the molecular mass sum from the measured ultrasound speeds as well as a unit (122) for displaying the measured values.

8. The device (10) according to claim 1, characterized in that it comprises (c) a second source (112) for the reference gas mix, which essentially has the same molecular mass sum as ambient air but a higher oxygen content than ambient air and contains no supplement of the specifically heavier gas.

9. The device (10) according to claim 1, characterized in that the device (12) for determining the molecular mass sum comprises a unit (123) for calculating the measured values for standard conditions.

10. The device (10) according to claim 1, characterized in that it comprises units (14; 140; 141; 142) for switching from the gas mix of ambient air (AA) inhaled by the patient (P) to the test gas mix delivered from source (11) and back; or for switching from the test gas mix from source (112) inhaled by the patient (P) to the test gas mix from source (11) and back.

11. A test gas mix for determining the ventilation homogeneity (VH) of a spontaneously breathing or artificially respirated patient, characterized in that the test gas mix is a breathable gas mix, which has essentially the same molecular mass sum (MMS) as ambient air (AA) or a reference gas mix, but differs from these by at least one added specifically heavier gas (I2).

12. The test gas mix according to claim 11, characterized in that the molecular mass sum of the test gas mix differs by less than 10%, preferably by less than 5%, more preferably by less than 2%, most preferably by less than 1% from the molecular mass sum of the ambient air (AA) or the reference gas mix.

13. The test gas mix according to claim 11, characterized in that the test gas mix has a molecular mass sum of 28 to 33 g/mol, preferably about 29 or 32, more preferably 28.85 or 32.0 g/mol.

14. The test gas mix according to claim 11, characterized in that the at least one specifically heavier inert gas (I2) in the test gas mix is selected from the group consisting of argon, neon, krypton, radon, xenon and SF₆ as well as mixtures thereof, preferably radon and SF₆ and more preferably SF₆.

15. The test gas mix according to claim 11, characterized in that the at least one specifically lighter inert gas is helium.

16. A method of using of a test gas mix according to claim 11 comprising using the test gas mix in the device of claim 1.

17. A method for determining the ventilation homogeneity (VH) of a spontaneously breathing or artificially respirated patient (P), characterized in that the patient:
inhales actively or passively and in a controlled manner a breathable test gas mix, which essentially has the same molecular weight sum (MMS) as ambient air or a reference gas mix, but differs from both by addition of at least one specifically heavier inert gas (I2) and a sufficient proportion of a specifically lighter gas (I1), in order to adjust the molecular mass sum of the test gas to the about same value of the molecular mass sum of ambient air or the reference gas, and that the molecular mass sum (MMS) of the test gas exhaled by the patient is determined in order to evaluate the ventilation homogeneity of the patient from the differences in the test gas mix inhaled and exhaled by the patient.

18. The method according to claim 17, characterized in that in a test phase preceding the active breathing or passive respiration with the test gas mix the patient actively or passively inhales and exhales normal air or reference gas mix in a controlled manner in order to normalize conditions of the test gas breathing.

19. The method according to claim 17, characterized in that the at least one specifically heavier inert gas (I2) in the test gas mix is selected from the group consisting of argon, neon, krypton, radon, xenon and SF₆ as well as mixtures thereof, preferably radon and SF₆ and more preferably SF₆.

20. The method according to claim 17, characterized in that the at least one specifically lighter inert gas is helium.

21. The method according to claim 17, characterized in that the molecular mass sum of the test gas mix differs by less than 10%, preferably by less than 5%, more preferably by less than 2%, most preferably by less than 1% from the molecular mass sum of the ambient air (AA) or the reference gas.

22. The method according to claim 17, characterized in that the gas mix used as reference gas comprises oxygen in a concentration of 21 to 100, preferably 30 to 100, more preferably 50 to 100, most preferably 100 vol.-%.

23. The method according to claim 17, characterized in that the test gas mix has a molecular mass sum of 28 to 33 g/mol, preferably about 29 to 32, more preferably 28.85 or 32.0 g/mol.

24. The method according to claim 17, characterized in that for determining the molecular mass sum (MMS) of the test gas mix exhaled by the patient (P) a measurement of the ultrasound speed in gases or gas mixtures is used and the measured ultrasound speeds are calculated into molecular mass sums.
